(19) United States
(12) Patent Application Publication
(10) Pub. No.: US 2018/0346130 A1
(43) Pub. Date: Dec. 6, 2018

(54) Cockpit and Crew Rest Air Quality Sensor

(71) Applicant: Astronics Advanced Electronic Systems Corp., Kirkland, WA (US)

(72) Inventor: Jeffrey A. Jouper, Newcastle, WA (US)

(21) Appl. No.: 15/992,197

(22) Filed: May 30, 2018

Related U.S. Application Data
(60) Provisional application No. 62/514,047, filed on Jun. 2, 2017.

(51) Int. Cl. B64D 13/06; G01N 33/00; G08B 21/14

(52) U.S. Cl. CPC B64D 13/06; G01N 33/0063; B64D 2013/0603; G01N 33/0047; G08B 21/14; G01N 33/004

(57) ABSTRACT

Disclosed is an area sensor for air quality in the cockpit, crew rest or other area of an aircraft. Noxious fumes, fuel vapors, carbon monoxide and other vapors can cause significant risks to the flight crew of an aircraft. Fumes may cause drowsiness, inattentiveness or confusion to the pilot of an aircraft placing all persons on board an aircraft in danger. There exists a need to monitor and warn the cabin crew of such an event so that measures can be taken, such as donning an oxygen mask, to mitigate the fumes. Many of the vapors encountered that can cause this issue are colorless and/or odorless and therefore not always detected by the flight crew especially if they are in sufficient quantities and exposure is long enough to compromise the pilots cognitive skills.

### CROSS-REFERENCE TO RELATED PATENT APPLICATION

This patent application claims a benefit to U.S. Provisional Patent Application Ser. No. 62/514,047, titled “Cockpit and Crew Rest Air Quality Sensor,” that was filed on Jun. 2, 2017. The disclosure of U.S. 62/514,047 is incorporated by reference herein in its entirety.

### BACKGROUND OF THE DISCLOSURE

Today, systems for measuring the air quality in an aircraft cabin are tied to an oxygen mask or to the Environmental Cooling System (ECS) that monitors Volatile Organic Compound (VOC) materials in ambient air. The ECS and VOC systems are electrically functional with the host system they are attached to. They control the airflow and warning systems at an aircraft level. Generally, the ECS and VOC systems are at the intake or exhaust of a pump system. All of the air circulated within an aircraft cabin, cockpit and crew rest areas comingle through a single sensor. Because sensors measure comingled air, the sensors take a gross measurement of all air within the aircraft environment. The systems ECS and VOC on an oxygen mask control the mixture of oxygen and other compounds to deliver quality air to a pilot or passenger during emergency situations. The embodiments disclosed herein differ from the typical centralized set of ducts used to monitor gross air quality such as U.S. Pat. No. 9,957,052B2 titled: “Aircraft environmental control system that optimizes the proportion of outside air from engines, APU's, ground air sources and the recirculated cabin air to maintain occupant comfort and maximize fuel economy,” which is incorporated herein by reference in its entirety.

Aircraft use air quality sensors, as described above, to monitor events such as outgassing of vapors from fuels, fluids and faulty electronics. This sensing technique measures comingled air through a cabin and does not isolate specific points of air quality degradation. Some gasses, such as carbon monoxide can be clear and odorless and may cause cognitive degradation in an aircraft flight crew if present in a high enough concentration. A pilot may make errors or suffer impaired judgment if carbon monoxide causes sensory degradation. Such, errors and impaired judgment may lead to catastrophic events. An aircraft system able to detect an exact gas source location would be particularly advantageous and aid in flight safety.

Active equipment events such as out-gassing of electronics, lithium batteries, etc. require quick notification as noxious fumes typically spread rapidly in these events. In a confined area such as a crew rest area, air quality degradation could happen in a matter of minutes causing breathing stress, loss of cognitive skills etc.

There remains a need for small modular sensor units that monitor local air quality around electronics close to the flight crew. Particularly, there is a need for such units in areas such as crew rest that may have limited air flow, and close proximity to sources of out-gassing electronics. Examples of outgassing sources may include a capacitor venting event or a lithium battery powered device in the early stages of battery failure. Monitoring and event warnings prevent possible aircraft flight safety disturbances, as the events can degrade cognitive ability in the pilot.

### SUMMARY OF THE DISCLOSURE

The present device is a battery powered or energy harvesting sensor that can be placed on any surface, behind a panel, near electronics, near the pilot or crew member. The sensor monitors air quality in real time and real location as needed. In embodiments, the sensor may contain a radio for transmitting the detected air quality to a data collection system. A data collection system may monitor the health of the ambient air around the sensor. The air quality data can be manipulated and sent to a storage and collection system for analysis either during flight or post-flight. Analysts can use this air quality data to better understand crew member risks in specific areas of the aircraft.

In embodiments, the present air quality sensor contains a detector effective to determine a pre-specified vapor concentration. The sensor also contains a power source and a microcontroller, each coupled to the detector. A housing encases the detector, and has an inlet and an outlet extending through it.

### BRIEF DESCRIPTION OF THE DRAWINGS

FIG. 1a shows a top view of a sensor assembly with no a pump.

FIG. 1b shows a top view of a sensor assembly with a pump.

FIG. 2a shows a cutaway side view and a top view of the sensor assembly with no pump.

FIG. 2b shows a cutaway side view and a top view of the sensor assembly with a pump included.

FIG. 3 shows a schematic of the sensor assembly.

FIG. 4 shows a cutaway side view of a piezo electric pump.

### DETAILED DESCRIPTION

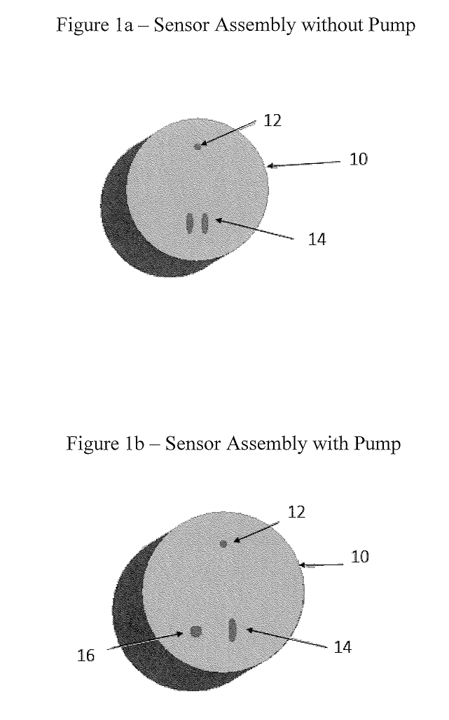

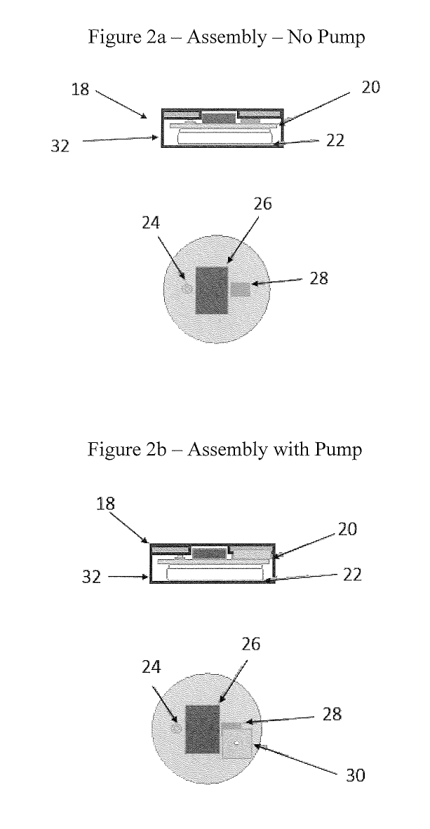

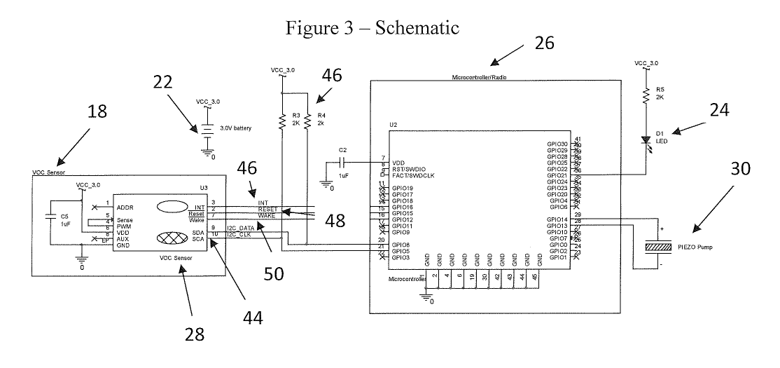

FIG. 1a depicts a sensor 10 that contains a sensor vent 14 enabling the sensor 10 to access ambient air within a cockpit, crew rest or any other area of an aircraft where sensing air quality is desired. The sensor 10 takes periodic samples of the surrounding air under the control of a microcontroller (uC) 26 (as illustrated in FIG. 2). The sensor 10 and the uC 26 communicate across a bi-directional Inter-Integrated Circuit (I2C) bus 44 (as illustrated in FIG. 3). The uC 26 sends a command string to wake the sensor 10, perform a measurement, then put the senor 10 back to sleep. Placing the sensor 10 in a low power mode conserves power reserves and facilitates powering the sensor 10 from energy harvesting in proper conditions. U.S. patent application Ser. No. 15/427,131, by Jouper, titled “Network System for Autonomous Data Collection,” describes powering sensors and communication networks by energy harvesting. The disclosure of U.S. Ser. No. 15/427,131 is incorporated by reference herein in its entirety.

In embodiments, the sensor 10 may contain a micro heat plate with a resistive element. The heat plate may reach a high temperature such as 350 C. As VOC elements contact the resistive element, the VOC reading changes value. These changes in value are correlated to parts per billion (ppb) air quality measurements as read by the sensor 10. The VOC sensor 28 can monitor for the presence of several organic compounds such as CO, CO2, and NO2. The VOC sensor 28 can also monitor for other reducing (CO) or oxidizing (NO2) gases and measure them. The sensor 10 may report Total Volatile Organic Compound (TVOC) level in ppb. This level is compared to a nominal level of TVOC such as 800 ppb. The level chosen is factored by the nominal TVOC level in an environment and when a level above this is noted, the sensor 10 can set the INT if it is above the threshold. The sensor, preferably, can store the value in non-volatile memory in the uC 26 for comparison on each reading. By storing the value in the uC 26, system functionality can be adjusted for different ambient levels based on the location of the sensor 10. The uC 26 may signify that air quality has significantly dropped and require intervention by the cabin crew when there is a significant change in the TVOC level.

During initialization, the uC 26 sets a VOC sensor 28 into a mode to periodically sample the air surrounding the sensor 10. Once set, the VOC sensor 28 performs the air quality sample at a particular rate. For example, a VOC sensor 28 may take air quality samples every 15 seconds. The period between samples relates to the area to be monitored, battery life required and system requirements. This period could be anywhere from milliseconds, when the sensor is adjacent active equipment, to once a minute when the sensor is in areas such as the crew rest.

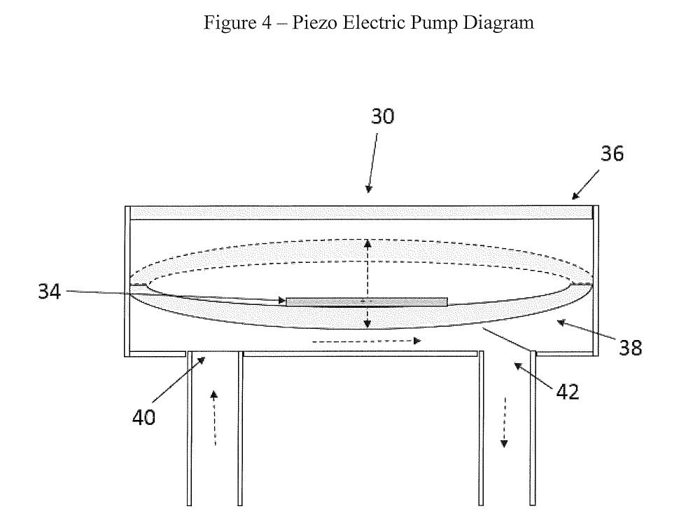

FIG. 1b depicts a pump 30 (such as a piezo electric air movement element as illustrated in FIG. 4. A representative example of a piezo electric air movement element is the Liquid/Gas Micro Pump by Curiejet) that may be used to move air through the sensor 10 and aid in local area air sampling. This process may be repeated as necessary to take adequate measurements. Moving air through the sensor will increase the sample area of the sensor 10 by drawing in air periodically just prior to sampling. The exchange of air within the sensor 10 enhances its capability to detect an event. However, this is not required in all instances of operation.

Should the pump 30 be used, the uC 26 may activate the pump 30 for a long enough period such as 10-1000 milliseconds prior to the sampling of the VOC sensor 28. This allows for the full exchange of the sampled air in the sensor 10. The pump 30 is located at an inlet 16 opening in an exterior wall 32 of the VOC sensor 28. When the pump 30 is activated, air is drawn in via the inlet 16 through an exterior wall 32 of the sensor housing 18. The exhaust of the pump 30 feeds air into the inlet 16 of the VOC sensor 28. A sensor vent 14 through the exterior wall 32 of the VOC sensor 28 exhausts air already in the sensor 10 through the housing 18 of the sensor 10. This allows for a full exchange of air already in the sensor 10 with air outside the sensor 10.

FIGS. 2a and 2b are assembly drawings of the sensor 10 with and without the pump 30 respectively. In these embodiments, the sensor 10 has an LED 24, a uC/Radio 26, a VOC sensor 28, an optional pump 30, and an exterior wall 32 enclosing each of these components together. Each of the components are connected in an electrical network. The uC/Radio 26 is connected to the VOC sensor 28 and the I2C bus 44. A battery 22 assembly is connected to the uC/Radio 26 to power the uC 26 and the VOC sensor 28.

FIG. 3 is a schematic diagram of an exemplary system. FIG. 3 shows a microcontroller and transmit/receive radio 26 within a single module. An interconnect between the microcontroller 26, micro pump 30 for air circulation and the VOC sensor 10 complete a system along with a battery 22 to power the system.

FIG. 3 further depicts an embodiment that includes the micro pump 30 for completeness, but the micro pump 30 is not required in every embodiment. FIG. 3 depicts a uC/Radio 26 connected to a sensor 10 to communicate with the sensor 10 over the I2C bus 44. FIG. 3 further depicts resisters R3 and R4 46, which provide pull ups to known states for embodiments that contain data and clock interfaces. INT 46, RESET 48, and WAKE 50 connections control the operational state of the sensor placing it into WAKE or SLEEP mode, RESET 48 can reset the sensor 10 should a firmware issue in the sensor 10 arise and the INT 46 is an interrupt output from the sensor 10 to signify that it has completed a measurement to the uC 26. An LED 24 is used as an optional indicator for power ON, operational state by flashing at a first rate of once per second, or fault by flashing at a second rate of twice per second as an example. The battery 22 provides operational power to all components on the schematic. The piezo pump 30 can optionally provide a method to move air through the VOC sensor 28 to increase sampling of air rather than waiting for the air exchange of time as would be done without the pump

FIG. 4 depicts an embodiment of a pump 30 that uses a piezo electric air movement element 34 coupled to a housing 36 and diaphragm 38. This pump allows the piezo electric air movement element 34 to move under electrical control to draw air in through an input port 40 when the piezo electric air movement element 34 is moved in a first direction and the expel air through an output port 42 when moved in a second direction. The piezo electric air movement element 34 movement direction is controlled by either positive or negative application of electrical current to the piezo air movement element 34.

1. A sensor comprising:
a detector effective to determine a vapor concentration;
a power source electrically interconnected to the detector;
a microcontroller in data communication with the detector;
a radio connected to the detector; and
a housing encasing the detector, the housing having an inlet and an outlet extending therethrough.

2. The sensor of claim 1 wherein the vapor concentration is pre-specified.

3. The sensor of claim 1 wherein the vapor is selected from the group consisting of volatile organic compounds (VOC) and carbon monoxide.

4. The sensor of claim 3 wherein the power source is selected from the group consisting of a battery and an energy harvester.

5. The sensor of claim 4 further including a pump effective to draw air into the housing through the inlet.

6. The sensor of claim 5 wherein the pump contains a piezo electric element.

7. The sensor of claim 6 wherein the pump has an outlet that is connected to the detector.

8. The sensor of claim 7 wherein the detector has an outlet that is connected to the outlet of the encasing.

9. The sensor of claim 8 wherein the detector is connected to a notification system.

10. The sensor of claim 9 wherein the detector is wirelessly connected to a notification system.

11. The sensor of claim 10 wherein the notification system is integrated into an aircraft.

12. A method of sensing air in an aircraft comprising moving air into a sensor with a pump;
analyzing the air with the sensor; and
outputting a set of analysis results.

13. The method of claim 12 further comprising: putting the sensor in a closed environment.

14. The method of claim 13 where the closed environment is an aircraft environment.

15. The method of claim 14 further comprising identifying pre-set composition limits.

16. The method of claim 15 further comprising sensing air composition limits.

17. The method of claim 16 further comprising storing air quality data.

18. The method of claim 17 further comprising notifying aircraft crew when sensed air composition exceed pre-set composition limits.

19. The method of claim 18 further comprising notifying a ground crew when sensed air composition exceeds pre-set composition.
